(19) United States
(12) Patent Application Publication
(10) Pub. No.: US 2017/0203096 A1
(43) Pub. Date: Jul. 20, 2017

(54) MULTI-LAYERED STRUCTURE AND METHOD

(71) Applicant: Heraeus Deutschland GmbH & Co. KG, Hanau (DE)

(72) Inventors: Stefan Schibli, Frankfurt am Main (DE); Jami A. Hafiz, Cedar Park, TX (US); Jens Troetzschel, Ronneburg-Neuwiedermus (DE)

(73) Assignee: Heraeus Deutschland GmbH & Co. KG, Hanau (DE)

(21) Appl. No.: 15/477,819

(22) Filed: Apr. 3, 2017

Related U.S. Application Data
(63) Continuation-in-part of application No. 14/991,307, filed on Jan. 8, 2016, now Pat. No. 9,636,026.
Application No. 14/991,307 is a division of application No. 14/323,163, filed on Jul. 3, 2014, now Pat. No. 9,498,142.

(51) Int. Cl. A61N 1/05; B21F 19/00

(52) U.S. Cl. CPC A61N 1/05; B21F 19/00; A61B 2562/125; A61M 2025/09108

(57) ABSTRACT

One aspect relates to a layered structure with a substrate, a first layer over the substrate, and a second layer over the first layer. The substrate and the second layer are an electrically conductive material and the first layer is an insulating material or the substrate and the second layer are insulating material and the first layer is electrically conductive material. At least one of the first and second layers includes an electrically conductive polymer.

### CROSS-REFERENCE TO RELATED APPLICATIONS

This application is a continuation-in-part of U.S. patent application Ser. No. 14/991,307, entitled “MULTI-LAYERED STRUCTURE,” having a filing date of Jan. 8, 2016, which is a divisional of U.S. Pat. No. 9,498,142, entitled “MULTI-LAYERED STRUCTURE AND METHOD,” having a filing date of Jul. 3, 2014, each of which are incorporated herein by reference.

### BACKGROUND

One aspect relates to a layered structure and another to a method for producing a layered structure. Prior methods describe layered structures having layers differing in conductivity which are usually produced through the use of laborious sputtering processes. This involves the use of complex equipment for maintenance of the process conditions and, in addition, expensive methods for vaporization of the materials used for sputtering. In general, it is desired to overcome, at least in part, the disadvantages resulting according to the prior art.

For these and other reasons there is a need for the invention.

### BRIEF DESCRIPTION OF THE DRAWINGS

The accompanying drawings are included to provide a further understanding of embodiments and are incorporated in and constitute a part of this specification. The drawings illustrate embodiments and together with the description serve to explain principles of embodiments. Other embodiments and many of the intended advantages of embodiments will be readily appreciated as they become better understood by reference to the following detailed description. The elements of the drawings are not necessarily to scale relative to each other. Like reference numerals designate corresponding similar parts.

FIG. 1 illustrates a side view of a layered structure according to one embodiment.

FIGS. 2a-2c illustrate respective length-wise and width-wise cross-sectional views of a layered structure according to various embodiments.

FIGS. 3a-3f are cross-sectional views illustrating a method of forming a layered structure according to one embodiment.

FIGS. 4a-4c are cross-sectional views illustrating a method of forming a layered structure according to one embodiment.

FIGS. 5a-5d are cross-sectional views illustrating a method of forming a layered structure according to one embodiment.

FIGS. 6a-6d are cross-sectional views illustrating a method of forming a layered structure according to one embodiment.

FIG. 7 illustrates an implantable medical system including a layered structure according to one embodiment.

FIGS. 8a-8d are cross-sectional views illustrating a method of forming a layered structure according to one embodiment.

FIG. 9 illustrates an implantable medical system including a layered structure according to one embodiment.

FIGS. 10a and 10b illustrate top and sides views of an implantable medical system including a layered structure according to one embodiment.

FIG. 11a illustrates a length-wise cross-sectional view of a layered structure according to one embodiment.

FIG. 11b illustrates a width-wise cross-sectional views of a layered structure according to one embodiment.

FIG. 11c illustrates a width-wise cross-sectional views of a layered structure, including an enlarged section of one layer, according to one embodiment.

FIG. 12 illustrates a schematic drawing of a detection unit according to one embodiment.

FIGS. 13a-13c illustrate schematically and exemplarily the piezoresistive material according to one embodiment.

FIG. 14 illustrates a particle size distribution for an exemplary carbon component.

FIG. 15 illustrates a pore size distribution for an exemplary carbon particle.

FIG. 16 illustrates a schematic overview of an electrical conductivity of a piezoresistive material depending on a carbon concentration.

FIG. 17 illustrates basic steps of an example of a method for producing a piezoresistive material according to one embodiment.

FIG. 18 illustrates a percolation threshold.

FIG. 19 illustrates the detected electric resistance for an increasing load.

FIG. 20 illustrates a detected electric resistance for a linear increase of blood pressure.

### DETAILED DESCRIPTION

In the following Detailed Description, reference is made to the accompanying drawings, which form a part hereof, and in which is shown by way of illustration specific embodiments in which the invention may be practiced. In this regard, directional terminology, such as “top,” “bottom,” “front,” “back,” “leading,” “trailing,” etc., is used with reference to the orientation of the Figure(s) being described. Because components of embodiments can be positioned in a number of different orientations, the directional terminology is used for purposes of illustration and is in no way limiting. It is to be understood that other embodiments may be utilized and structural or logical changes may be made without departing from the scope of the invention. The following detailed description, therefore, is not to be taken in a limiting sense, and the scope of the invention is defined by the appended claims.

It is to be understood that the features of the various exemplary embodiments described herein may be combined with each other, unless specifically noted otherwise.

One embodiment provides an inexpensive and efficient method for producing a layered structure of at least three layers.

One embodiment provides a method for producing a layered structure of at least three layers that can be used to produce layers that are as thin as possible.

One embodiment provides a simple and rapid method for producing a layered structure of at least three layers of which at least two layers differ in conductivity, that is, of which at least one layer is electrically conductive and at least one second layer is insulating.

One embodiment provides a layered structure of at least three layers that is simple and inexpensive to produce.

One embodiment provides a measuring device having a layered structure, whereby the layered structure is at least as accurate, reliable or long-lasting in use as the measuring devices with layered structures known according to the prior art.

In one embodiment, “electrically conductive” means that the object referred to as being electrically conductive has a specific sheet resistance of less than 10 kΩ (10,000 Ohm), in one embodiment less than 5 kΩ or in one embodiment less than 1 kΩ In many cases, the specific sheet resistance is concurrently more than 1Ω, in one embodiment more than 5Ω, though. If the substrate includes multiple electrically conductive layers, the specific sheet resistance of each of the layers meets at least one of the preceding criteria. Several of the multiple electrically conductive layers can have the same or different sheet resistance values within the range of the criteria specified above.

In one embodiment, “insulating” or “insulative” mean that the object referred to as being insulating has a specific sheet resistance of more than 50 kΩ, in one embodiment more than 500 kΩ or in one embodiment more than 1 MΩ (1,000,000 Ohm). In many cases, the specific sheet resistance is concurrently less than 100 MΩ, in one embodiment less than 10 MΩ, though. If the substrate includes multiple electrically conductive layers, the specific sheet resistance of each of said layers meets at least one of the preceding criteria. Several of the multiple electrically conductive layers can have the same or different sheet resistance values within the range of the criteria specified above.

In one embodiment, “biocompatible” means that the object referred to as being biocompatible meets the pertinent biocompatibility requirements according to the ISO 10993 1-20 standard.

According to one embodiment, a method for producing a layered structure includes providing a substrate;

forming a first layer onto at least part of the substrate, the first layer being a first polymer;

forming a second layer onto at least part of the first layer, the second layer being a second polymer;

wherein the substrate and the second layer are electrically conductive and the first layer is insulating or the substrate and the second layer are insulating and the first layer is electrically conductive; and

characterized in that forming each of the first and second layers includes forming such that each of the first and second layers is no more than one tenth of the thickness of the substrate.

In one embodiment, at least one of the first and second layers includes an electrically conductive PEDOT material. In one embodiment, forming each of the first and second layers includes forming such that each of the first and second layers is no more than one fiftieth of the thickness of the substrate. In one embodiment, forming of at least one of the first and second layers includes masking ends of the layer to define contact areas that are configured for attaching an electrically conducting contact. In one embodiment, forming of at least one of the first and second layers includes using a conductive PEDOT polymer followed by using a chemical etch to change at least portions of the layer from conductive to insulative. In one embodiment, the method includes forming a third layer over the second layer, the third layer comprising an insulative photoresist material.

In the embodiments, the conductive polymers provide conductivity in a layer that is very thin, provides great flexibly and elongation, and that is easy to manufacture at relatively low cost. Furthermore, the embodiments significantly maintain the base shape, size, appearance and configuration of the substrate.

In one embodiment, a layered structure includes a substrate, a first layer over the substrate, and a second layer over the first layer. The substrate and the second layer are an electrically conductive material and the first layer is an insulating material or the substrate and the second layer are insulating material and the first layer is electrically conductive material, and that at least one of the first and second layers includes an electrically conductive polymer. In one embodiment, at least one of the first and second layers includes electrically conductive PEDOT. In one embodiment, each of the first and second layers are one tenth of the thickness of the substrate. In one embodiment, the layered structure has a length defining first and second ends and wherein at least one of the first and second layers extends substantially the length of the layered structure and includes contact areas proximate to the first and second ends that are configured for attaching an electrically conducting contact. In one embodiment the thickness of at least one of the first and second layers is in a range from 0.05 μm to 10 μm. In one embodiment, the substrate includes one of a metal wire and metal insertion needle and at least one of the first and second layers is electrically conductive PEDOT. In one embodiment, the substrate and the first and second layers are biocompatible such that the layered structure is configured for implantation into the human body. In one embodiment, the substrate has a sheet resistance of less than 10 kΩ.

In the embodiments, the conductive polymers provide conductivity in a layer that is very thin, provides great flexibly and elongation, and that is easy to manufacture at relatively low cost. Furthermore, the embodiments significantly maintain the base shape, size, appearance and configuration of the substrate.

In one embodiment, a layered structure includes a conductive substrate, a first layer over the substrate, wherein the first layer includes an insulating material and includes at least one access area, and a contact area at least partially within the access area and in conductive contact with the substrate. The first layer has a thickness that is no more than one tenth of the thickness of the substrate. In one embodiment, the contact area is an electrically conductive PEDOT material. In one embodiment, the access area is a hole in the first layer having a diameter of less than 0.4 mm. In one embodiment, each of the first layer and the contact area are no more than one tenth the thickness of the substrate. In one embodiment, the substrate, the contact area and the contact area are biocompatible such that layered structure is configured for implantation into the human body. In one embodiment, the thickness of the first layer is no more than one fiftieth of the thickness of the substrate.

The described embodiments can achieve very small contact areas within the first layer, which is itself very thin. This provides great flexibly and elongation, and is easy to manufacture at relatively low cost. Furthermore, the embodiments significantly maintain the base shape, size, appearance and configuration of the substrate.

In one embodiment, a piezoresistive material is used. The piezoresistive material includes a compound of a carbon component and an elastomer component. The carbon component includes carbon particles comprising macropores. The elastomer component includes polymeric chains. At least some of the macropores in the carbon particles are infiltrated by polymeric chains to form a piezoresistive interconnection between the carbon particles.

The term “piezoresistive” may be understood in that the piezoresistive material is subjected to a change of its electrical resistivity when mechanical stress is applied to the piezoresistive material. The mechanical stress may be an elastic, isostatic or unidirectional compressive load. The mechanical stress may be at least one of a group comprising force, pressure, motion, vibration, acceleration and elongation.

The term “piezoresistive interconnection” may be understood in that the carbon component and the elastomer component are interconnected to form a compound material which has a piezoresistive effect. This means, when mechanical stress is applied to the compound of carbon component and elastomer component, the compound illustrates a change of its electrical resistivity and in particular a decrease of its electrical resistivity and an increase of electrical conductivity.

In one embodiment, the interconnection between the carbon component and the elastomer component includes an infiltration of polymeric chains of the elastomer component into the macropores within the carbon particles.

The dimensions of the macropores of the carbon particles may therefore be adapted to the dimensions of polymeric precursors of the elastomer component. This means, the diameter of a polymer emulsion particle is in a range of a diameter of a macropore. The interconnection may further include that at least some of the carbon particles are linked by polymeric chains. Such rigid mechanical interconnection between carbon particles and polymeric chains enables a most complete geometrical restoring after elastic compression of the material.

In one embodiment, the piezoresistance of the interconnection is based on the fact that the polymeric chains between the carbon particles of the carbon component rearrange and relax when the piezoresistive material is subjected to a compressive load. The rearrangement and relaxation enables a formation of electrical paths between the electrically conductive carbon particles and consequently reduces the electrical resistance of the piezoresistive material.

In other words, the piezoresistive material according to one embodiment may be a material comprising elastomer filled with porous carbon particles to form part of a resistive sensor which shows a negative change of electrical resistance when subjected to pressure.

As a result, one embodiment refers to a piezoresistive material with at least one of the following advantages: a possibility to form piezoresistive devices showing a superior sensitivity, a possibility to form very small piezoresistive devices, a possibility to form flexible piezoresistive devices, and a possibility to form a piezoresistive device with a large measuring or detection range, a small dependence on temperatures and/or a very good relaxation behavior. Further, the piezoresistive material according to one embodiment allows an easy and cheap manufacture, may be manufactured in all kinds of shapes and sizes (for example, by 3D and conventional printing, drawing, molding, injection molding, painting, spraying, screen printing, coating etc.) and may be adapted during manufacture in view of its elastic modulus, flexibility etc. by, for example, tuning the physical properties of the elastomer component.

The term “carbon component” may be understood as a component comprising carbon particles with open porosity and macropores. The term “macropores” may be understood as pores having a size between 50 and 1000 nm measured by, for example, Hg porosimetry. The carbon particles may be highly porous. The term “highly porous” may be understood as having a total pore volume between 0.7 and 3.5 cm3/g, and in one embodiment between 0.9 and 2.5 cm3/g.

The term “elastomer component” may be understood as a component comprising an elastomer, which is an elastic polymer. The molecular structure of elastomers can be imagined as a ‘spaghetti and meatball’ structure, with the meatballs signifying cross-links. Elasticity is derived from an ability of long chains to reconfigure themselves to distribute an applied stress. Covalent cross-linkages ensure that the elastomer will return to its original configuration when the mechanical stress is removed.

The term “polymeric chains” may be understood as covalently bonded links between monomers forming a network. The polymeric chains may block an electric conductivity between the carbon particles in an unloaded condition of the piezoresistive material. When a load, as, for example, mechanical pressure, is applied to the piezoresistive material, the polymeric chains may be compressed and the electrically conductive carbon particles may contact each other to implement an electric conductivity of the piezoresistive material.

The term “infiltrated” may be understood in that polymeric chains penetrate into pores of the carbon particles. The polymeric chains may also penetrate through pores of carbon particles and thereby link several carbon particles to each other.

In an example, the macropores in the carbon particles have a macropore volume between 0.6 and 2.4 cm3/g calculated from pores sizes ranging from 50 to 1000 nm, and in one embodiment between 0.8 and 2.2 cm3/g. This large macropore volume enables a filling by the polymeric chains of the elastomer component and thus a fixation of the polymeric chains.

In an example, the carbon particles further include mesopores with a mesopore volume between 0.05 and 0.2 cm3/g, and in one embodiment between 0.1 and 0.15 cm3/g. The term “mesopores” may be understood as pores having a size between 2 and 50 nm measured by, for example, Hg porosimetry.

In an example, the carbon component is graphitized. The term “graphitized” may be understood in that a formation of graphitic carbon is initiated by an exposure to elevated temperatures between, for example, 1400 to 3000° C. During graphitization, micropores tend to disappear, mesopores rearrange and macropores remain constant. The result is a graphitized, porous carbon component comprising carbon particles with a large amount of macropores. The macropores can be linked with each other. The formation of graphite in the carbon component leads to an increased electrical conductivity. The graphitizing of the carbon component may here be done between 1400 and 3000° C., in one embodiment between 2300 and 2600° C.

In an example, the carbon component is graphitized to a graphitization degree between 60 and 80%, and in one embodiment to a graphitization degree of over 70%. The graphitization degree g may be calculated based on a measured distance d002 of graphite basal levels: g=(344 pm−d002)/(344 pm-335.4 pm). A small distance d002 value thereby relates to a high graphitization degree.

In an example, the carbon particles include essentially no micropores which may be understood as having a micropore volume of less than 0.01 cm3/g. The term “micropores” may be understood as pores having a size smaller 2 nm measured by nitrogen adsorption (BET).

In an example, the amount of the carbon component in the elastomer component is near a percolation threshold. Near in the meaning of within the area of the percolation threshold, only few conductive paths exist in the piezoresistive material when not subjected to a load. However, if a load is applied to the piezoresistive material, the elastomer component is compressed and the electrically conductive carbon particles get in contact with each other. Further conductive paths appear, which thus increase the electrical conductivity of the piezoresistive material. As a result, near the percolation threshold, the sensitivity for pressure is extremely high. Outside the area of the percolation threshold, there is no sudden change of the electrical conductivity of the piezoresistive material.

In an example, the amount of the carbon component in the elastomer component is between 1 to 30 wt.-%, in one embodiment between 15 and 26 wt.-%.

In an example, the carbon particles have sizes d50 between 1 and 100 μm, in one embodiment between 5 and 20 μm.

In an example, only pores larger than a filling threshold are infiltrated by polymeric chains. Exemplarily, the filling threshold is between 60 and 250 nm, and in one embodiment between 60 and 150 nm.

In an example, the carbon component has a real density between 1.6 and 2.26 g/cm³, and in one embodiment between 2.0 and 2.26 g/cm³ as measured by He pycnometry.

In an example, the carbon component has a specific surface between 5 and 500 m2/g, and in one embodiment between 10 and 70 m2/g. The specific surface is here measured according to BET (Brunauer-Emmett-Teller).

In an example, the elastomer component includes rubber and/or silicone. Rubber may be styrene butadiene rubber, ethylene propylene diene monomer rubber or the like. The silicone of the elastomer component may have a viscosity in an uncured state between 10 Pa s and 2000 Pa s when measured, for example, according to DIN53019.

According to one embodiment, also a detection unit is presented. The detection unit includes a detection element and a processing element. The detection element includes the piezoresistive material as described above. The detection element may be a probe, a catheter tip, a blood pressure sensor, an artificial skin component or the like. The processing element is configured to process a decrease of electrical resistance detected by the piezoresistive material into a value of compressive load applied to the piezoresistive material. The processing element may be an analog digital converter.

In one embodiment, also several uses of the material or the detection unit as described above are presented. In general, the material or the detection unit may replace all kinds of elastomer components in all technical fields without influencing the mechanic behavior.

In an example, the material or the detection unit is used for a probe to detect a force, pressure, motion and/or vibration of the probe relative to a surrounding medium. Further, a detection of a change in force, pressure, motion, vibration etc. is possible. In addition, a detection of acceleration or elongation or their changes is possible. The surrounding medium may be gaseous, liquid or solid. It may be bone, tissue, organs, blood and/or the like. When using several probes, also a detection of a position of an occurrence or a change in force, pressure, motion, vibration etc. is possible.

In an example, the probe is a catheter tip configured to detect a force. The force may be in a range of 0.02 N to 10 N. Such catheter tip may be used to avoid harming the surrounding medium when moving a (balloon) catheter, for example, through blood vessels to assist to a navigation of the catheter through the surrounding medium etc.

Exemplarily, the probe is part of an ablation electrode to allow a better control of the ablation parameters.

In an example, the probe is a blood pressure sensor configured to detect a blood pressure. The blood pressure may be in a range of 40 mmHg to 200 mmHg. Such blood pressure sensor may be used to, for example, assist a pacemaker during adjustment or operation or to characterize a vascular constriction.

Exemplarily, the probe is configured to detect movements of organs, as, for example, a lung and/or a heart, for example, to detect sleep apnea. Exemplarily, the probe is configured to be used as strain sensors for, for example, human motion detection.

In an example, the probe is an artificial skin, muscle or hair component. For example, the skin component may be configured to detect a tactile sensation.

Exemplarily, the probe is part of a smart textile or a clothing as, for example, socks for diabetics or clothes for pulse measuring. In all cases, the probe may be applied to an, for example, elastomeric substrate by, for example, extrusion, screen printing or by means of a doctor blade. Exemplarily, the probe is part of a pulse measuring device of, for example, a smart watch.

Exemplarily, the probe is part of a wearable flexible stretch sensor. Exemplarily, the probe is part of a stress gauge for detecting stain applied to, for example, bones and in particular to feet. The probe may be integrated into sports equipment as, for example, a football or ice hockey helmet to detect, for example, a severity of a head impact.

Exemplarily, the probe is part of a haptic sensor for, for example, a gripping instrument.

In one embodiment, also a method for producing a piezoresistive material is presented. It includes the following steps:

a) mixing an elastomer component and a carbon component into a mixture, wherein the elastomer component includes polymeric chains and the carbon component includes carbon particles comprising macropores, and

b) curing the mixture so that at least some of the macropores in the carbon particles are infiltrated by polymeric chains to form a piezoresistive interconnection between the carbon particles.

The elastomer component may be liquid or an emulsion and may be made of at least one, and in one embodiment, two liquid subcomponent(s).

In an example, the method further includes a step of graphitizing the carbon component between 1400 and 3000° C., in one embodiment between 2300 and 2600° C. In the example, the graphitizing step is introduced before the mixing step.

Exemplarily, the method further includes a step of forming the mixture into a predefined shape of a product to be manufactured by means of, for example, extrusion or screen printing. This may be done before the curing step. The curing may be done for, for example, 4 hours at 200° C. Exemplarily, the method further includes a step of electrically contacting the cured product.

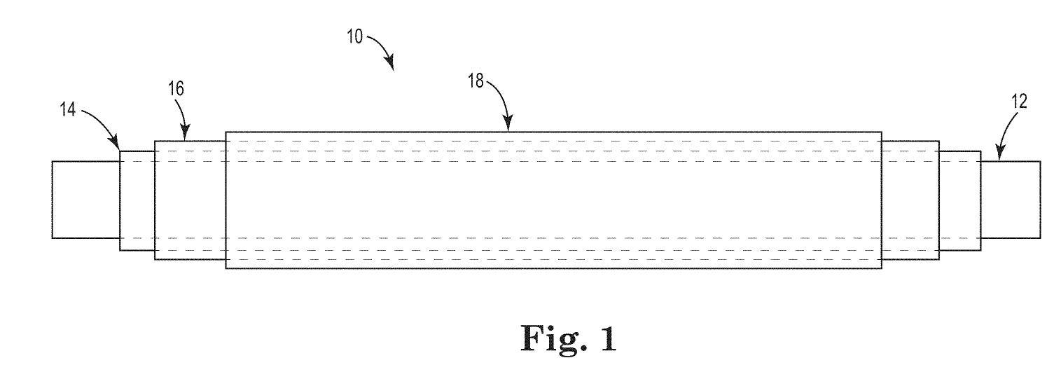

FIG. 1 illustrates a layered structure 10 in accordance with one embodiment. In one embodiment, layered structure 10 includes substrate 12, first layer 14, second layer 16 and third layer 18. In one embodiment, substrate 12 is conductive metal medical component, such as a wire, which is also biocompatible. First layer 14 is insulating layer such as an insulative polymer, which is also biocompatible, that is formed over substrate 12. Second layer 16 is a conductive layer, such as a conductive polymer, which is also biocompatible, that is formed over first layer 14. Finally, third layer 18 is an insulating layer, such as an insulative polymer, which is also biocompatible, that is formed over second layer 16.

As such, in one embodiment layered structure 10 is a fully biocompatible medical device that is useful for implantation or insertion in the human body. Furthermore, with its alternating conductive and non-conductive layers, it is useful in many applications where multiple independent conductors are needed in a small package that does not significantly alter the base component, such as substrate 12. In this way, layered structure 10 provides functional capability in multiple layers, yet layered structure 10 significantly maintains the base shape, size, appearance and configuration of substrate 12.

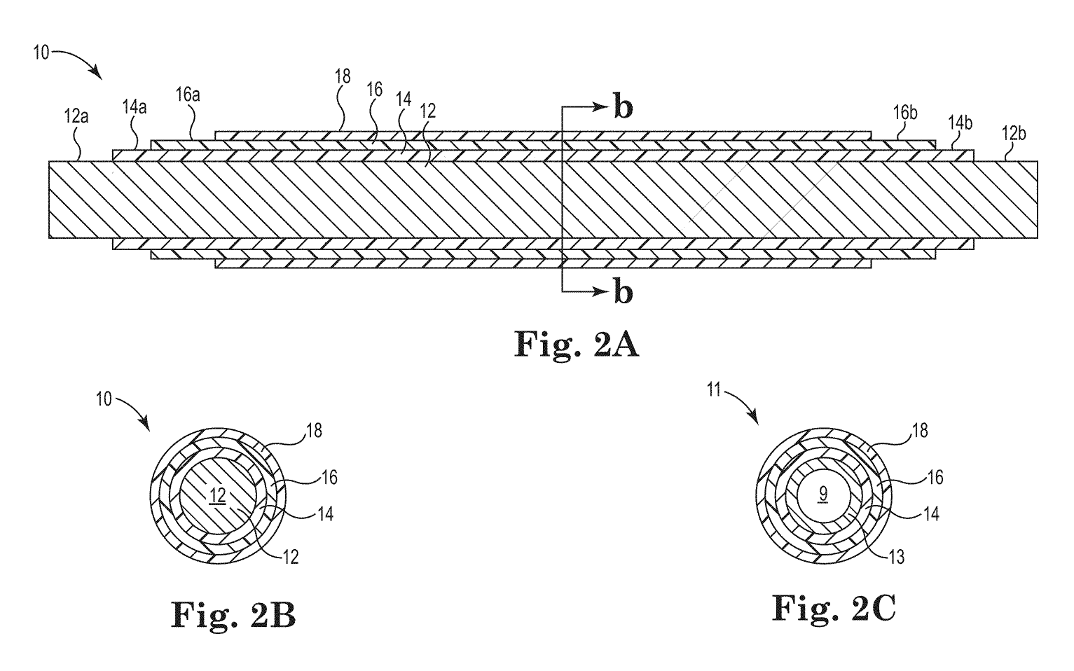

FIG. 2a illustrates a sectional view of layered structure 10 taken along its length, in accordance with one embodiment. FIG. 2b illustrates a sectional view of layered structure 10 from the line b-b in FIG. 2a. Layered structure 10 includes substrate 12, first layer 14, second layer 16 and third layer 18. As is apparent in the sectional view of FIG. 2a, in one embodiment, the ends of each of substrate 12 and layers 14, 16, and 18 are slightly staggered relative to each other. As such, substrate 12 includes exposed first and second contact areas 12a and 12b, which extend laterally beyond first layer 14 at opposite ends of layered structure 10. Similarly, second layer 16 includes exposed third and fourth contact areas 16a and 16b, which extend laterally beyond third layer 18 at opposite ends of layered structure 10.

First and second insulating areas 14a and 14b respectively insulate the area between first and third contact areas 12a and 16a and the area between second and fourth contact areas 12b and 16b. In one embodiment, sensors, measuring devices, wires or other conductive contacts can be attached to contact areas 12a, 12b, 16a and 16b as will be further discussed below. First and third layers 14 and 18 isolate conductive substrate 12 and conductive second layer 16 so that independent signals can be carried via layered structure 10 from first contact area 12a at one end to second contact are 12b at the other end (via substrate 12) and from third contact area 16a at one end to fourth contact are 16b at the other end (via second layer 16). Because layers 14, 16, and 18 are very thin and flexible, layered structure 10 significantly maintains the base shape, size, appearance and configuration of substrate 12.

In one embodiment, substrate 12 of layered structure 10 illustrated in FIGS. 2a and 2b is a solid wire, such as a medical guide wire or the like that can be implanted or temporality inserted into a human body. In the embodiment illustrated in FIG. 2c, layered structure 11 includes a substrate 13 that is a wire with a lumen 9 running through its center. Layers 14, 16, and 18 are added as described above with respect to layered structure 10. As such, layered structure 11 can also be used in medical implant situations, and further provides the ability to transmit material, such as medicine or a bodily fluid, via lumen 9.

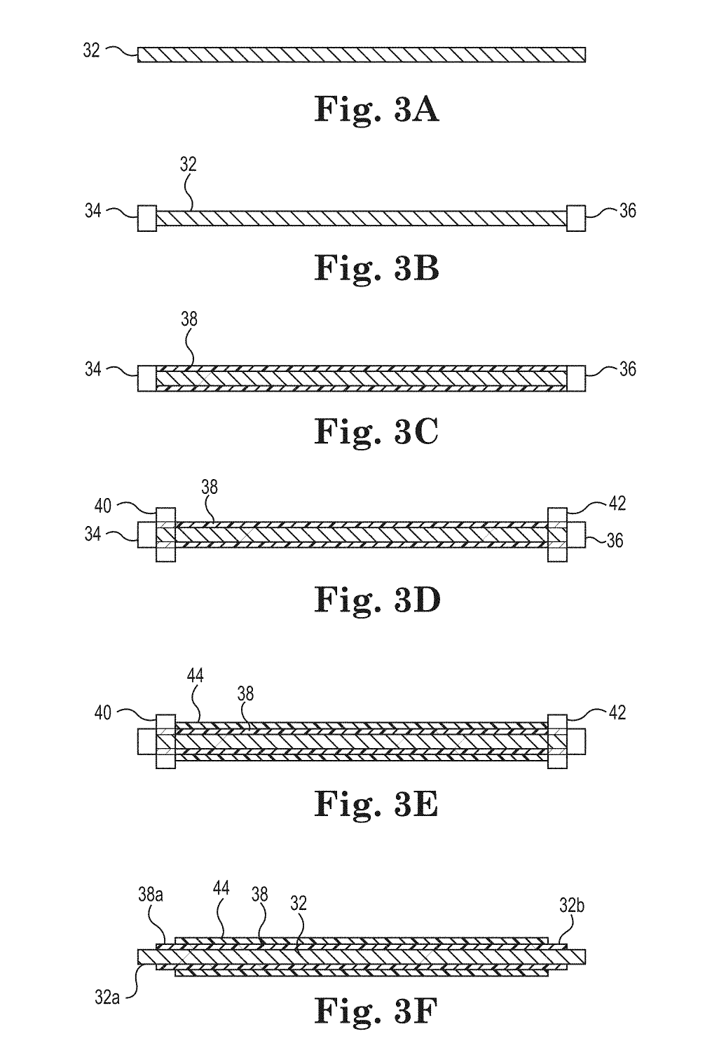

FIGS. 3a-3f illustrate sectional views of a method of forming a layered structure 50 (FIG. 3f) in accordance with one embodiment. A substrate 32 is illustrated in FIG. 3a. Substrate 32 is biocompatible and conductive, such as a medical wire. In FIG. 3b, first and second mask ends 34 and 36 are placed over substrate 32 at each of its respective ends. Masks 34 and 36 can be in a variety of forms, such as tape masks or a heat-shrink tube. In one embodiment, a polyimide tape is used for masks 34 and 36.

With masks 34 and 36 in place over substrate 32, first layer 38 is formed, as illustrated in FIG. 3c. In one embodiment, first layer 38 is a parylene coating that is a conductive insulator providing conductive isolation to substrate 32, except where first and second mask ends 34 and 36 prevented the formation of first layer 38. In one example, the parylene coating of first layer 38 is applied to substrate 32 at about 25 degrees C. under a pressure of about 0.1-1.0 Torr.

Next, third and fourth masks 40 and 42 are placed over the ends of first layer 38 immediately adjacent first and second masks 34 and 36, respectively, as illustrated in FIG. 3d. Masks 40 and 42 can also be in a variety of forms, such as tape masks, for example a polyimide tape, or a heat-shrink tube. With masks 40 and 42 in place, a second layer 44 is formed over first layer 38 between masks 40 and 42 as illustrated in FIG. 3e. In one embodiment, second layer 44 is a conductive layer, such as a conductive polymer.

In one embodiment, second layer 44 is a conductive polymer Poly(3,4-ethylenedioxythiophene) poly(styrenesulfonate), or PEDOT:PSS (hereinafter referred to as “PEDOT”), such as for example, a conductive polymer sold under the trade name CLEVIOS™. Second layer 44 may be applied in a variety of ways, for example, with deposition by in-situ polymerization, by printing or screen-printing, by spin coating, or by other known methods of application. In other embodiments, conductive polymers such as carbon nanotube conductive polymers or other conductive polymers can be used. In each case, the conductive polymers provide conductivity in a layer that is very thin, provides great flexibly and elongation, and that is easy to manufacture at relatively low cost.

Once first and second masks 34 and 36 and third and fourth masks 40 and 42 are removed, layered structure 50 is formed as illustrated in FIG. 3f As illustrated there, first and second contact areas 32a and 32b are located on the substrate 32 at the respective ends of layered structure 50, and will be accessible for attachment of sensors or other devices as described above with respect to layered structure 10. In addition, further layers can be readily added to layered structure 50 such that 2, 3, 4 or more contact areas with respective any added conductive layers are readily available for coupling sensors, measuring devices, wires or other conductive contacts useful in invasive or implantable medical devices.

In one embodiment of layered structure 50, substrate 32 is metal needle or wire having an outer diameter of about 0.5 mm. In other applications, substrate 32 may have a more rectangular shape, but have a thickness of about 0.5 mm. In some of the applications of such a needle, wire or similar device, it is important that independently accessible conductive contact areas are available on the needle or wire (such as contact areas 12a and 16a described above with respect to layered structure 10) for attaching sensor, measuring devices or the like. But, in some applications it is also important that the outer diameter or outer thickness of layered structure 50 does not significantly depart from the outer diameter or thickness substrate 32. Accordingly, in one embodiment, each of first layer 38, second layer 44, and any additional insulating and conductive layers added over them are orders of magnitude more thin than the outer diameter or thickness of substrate 32.

In one embodiment, each of first layer 38, second layer 44 and any additional insulating and conductive layers added over substrate 32 are no more than 50 microns, and in one embodiment each are between 2 and 10 microns. In such case, where substrate 32 is 0.5 mm in diameter or thickness and all subsequent added layers over it are no more than 50 microns, each added layer is no more than one tenth in thickness relative to the diameter or thickness of substrate 32. With each of these subsequent layers over substrate 32 having such relatively small thicknesses, the overall size of layered structure 50 does not significantly depart from that of substrate 32. This can be important in many medical applications where the size and shape of substrate 32, such as where it is an injection needle, must be kept limited in order for it to reach its targeted location without interfering or harming closely adjacent locations.

In addition, in one embodiment each of first layer 38, second layer 44, and any additional insulating and conductive layers over substrate 32 are each polymer layers. As such, unlike metallic coated layers, for example, each subsequent layer over substrate 32 is extremely flexible. This provides flexibility to layered structure 50 not available in prior devices. In one embodiment, each subsequent layer over substrate 32 is a polymer material that can sustain elongation of up to 300% without damaging the material, causing splits, or otherwise compromising the layers. By comparison, layers that are metallic-based and related coatings often fail after elongation on only 25%. Accordingly, layered structure 50 with polymer layers over substrate 32 is a flexible structure useful in many medical applications.

Furthermore, because layered structure 50 uses a conductive polymer coating like PEDOT and an insulative coating like parylene, both of which are biocompatible, it is readily useable as a device that is either inserted or implanted into a human body. Some metallic and other related coatings can react with blood and body fluids and partially dissolve components into the blood. Because the insulative and conductive coatings of layered structure 50 are biocompatible polymers, it will not be absorbed into the body.

In addition, because layered structure 50 uses a conductive polymer coating like PEDOT and an insulative coating like parylene, both of which can be kept extremely thin, yet at the same time not risk having pin holes, such as can be the case when metallic coatings are used in very thin layers. Such pin holes that occur with metallic coatings will cause subsequent layers to flow into the pin holes and jeopardize the integrity of the layers.

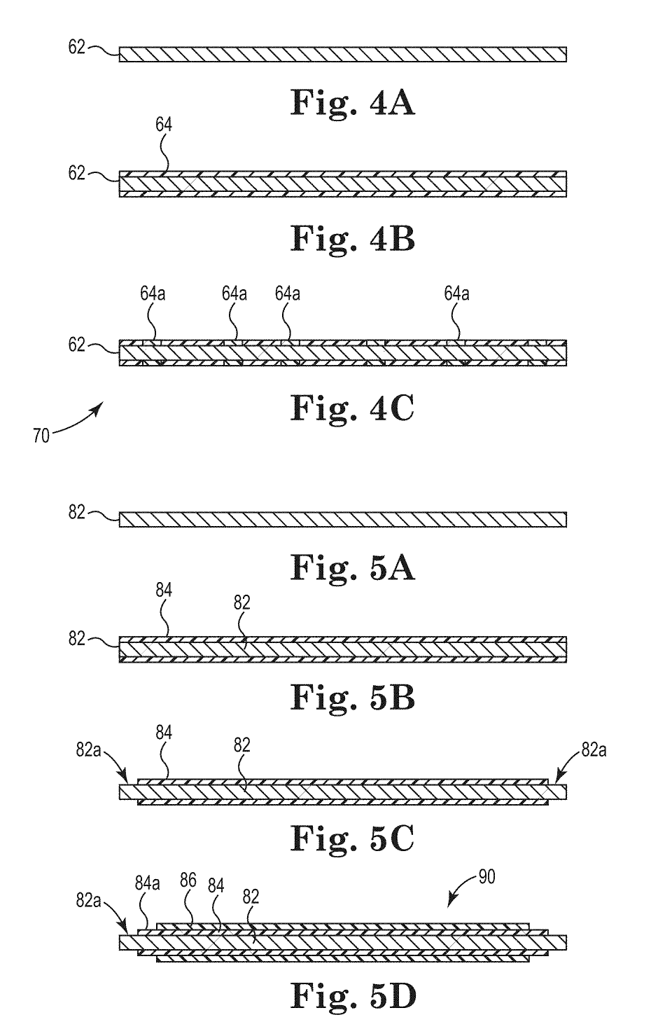

FIGS. 4a-4c illustrate sectional views of a method of forming a layered structure 70 in accordance with one embodiment. A substrate 62 is illustrated in FIG. 4a. Substrate 62 is biocompatible and conductive, such as a medical wire. In FIG. 4b, a first layer 64 is formed over substrate 62. In one embodiment, first layer 64 is a conductive layer, such as a PEDOT conductive polymer. For example, first layer 64 is a conductive polymer sold under the trade name CLEVIOS™. First layer 64 is conductive and may be applied in a variety of ways, for example, with deposition by in-situ polymerization, by screen-printing, or by other known methods of application.

Once first layer 64 is applied it can be selectively treated with CLEVIOS™ Etch, which destroys the conductivity of the CLEVIOS™ conductive polymer in the areas where the CLEVIOS™ Etch is applied. FIG. 4c illustrates isolating areas 64a where CLEVIOS™ Etch has been applied thereby rendering those areas 64a as non-conductive and also thereby defining areas of conductivity in first layer 64 between the isolating areas 64a. As such, layered structure 70 provides multiple conductive contact areas that are readily available for coupling sensors, measuring devices, wires or other conductive contacts useful in invasive or implantable medical devices.

Since CLEVIOS™ Etch can be readily applied by printing, the areas of conductivity and insulating properties can be precisely defined in a layer without need for masking. Avoiding masking allows for automation in manufacturing processes, which leads to significant cost savings in production. As with the previous process, more layers can be added to create unique multilayer devices with various configurations of conductivity and insulating properties between layers and even within a single layer of the device.

FIGS. 5a-5d illustrate sectional views of a method of forming a layered structure 90 in accordance with one embodiment. A substrate 82 is illustrated in FIG. 5a. Substrate 82 is biocompatible and conductive, such as a medical wire. In FIG. 5b, first layer 84 is formed over substrate 82. In one embodiment, first layer 84 is a Poly(methyl methacrylate) (PMMA) coating that is a conductive insulator providing conductive isolation to the substrate 82. In one example, the PMMA coating of first layer 84 is dip coated to cover substrate 82.

Next, first and second contact areas 82a and 82b are exposed on the substrate 82 by laser ablation of first layer 84 in those areas, as illustrated in FIG. 5c. In FIG. 5d, a second layer 86 is formed over first layer 84. In one embodiment, second layer 86 is a conductive polymer, such as PEDOT. In another embodiment, second layer 86 is a nano gold coating that is applied via inkjet printing. In such case, printing allows exact dimensions so that no masking is needed in forming layered structure 90. This allows for the use of automation in manufacturing, which leads to significant cost savings in production.

As illustrated in FIG. 5d, first and second contact areas 82a and 82b are located on the substrate 82 at the respective ends of layered structure 90, and will be accessible for attachment of sensors or other devices as described above with respect to layered structure 10. In addition, further layers can be readily added to layered structure 90 such that 2, 3, 4 or more contact areas with respective conductive layers are readily available for coupling sensors, measuring devices, wires or other conductive contacts useful in invasive or implantable medical devices.

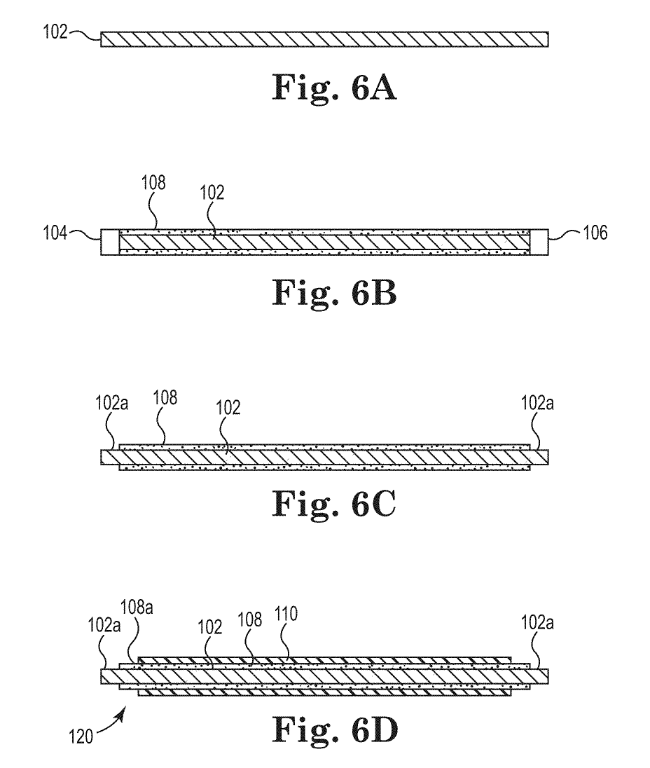

FIGS. 6a-6d illustrate sectional views of a method of forming a layered structure 120 (FIG. 6d) in accordance with one embodiment. A substrate 102 is illustrated in FIG. 6a. Substrate 102 is biocompatible and conductive, such as a medical wire. In FIG. 6b, first and second mask ends 104 and 106 are placed over substrate 102 at each of its respective ends. Masks 104 and 106 can be in a variety of forms, such as photomasks or other masks typically used in photolithography processes.

With masks 104 and 106 in place over substrate 102, first layer 108 is formed, as illustrated in FIG. 6b. In one embodiment, first layer 108 is a photoresist layer that is sputtered onto substrate 102, or otherwise applied. In one embodiment, first layer 108 is SU-8 photoresist, and is accordingly biocompatible and insulative. In FIG. 6c, first layer 108 is exposed to a UV source or laser such that the portion on first layer 108 left behind provides an insulating layer over substrate 102, except at the contact areas 102a, where masks 104 and 106 were located and then removed. In addition, a photomask can be used in conjunction with a photoresist layer as first layer 108, in order to selectively coat substrate 102. For example, a photomask can be used so that only sections of first layer 108 will be insulative, such as the layer illustrated in FIG. 4c. Then, conductive material can be formed in between these insulative sections, also as illustrated in FIG. 4c.

A second layer 110 is then formed over first layer 108 as illustrated in FIG. 6d to form layer structure 120. In one embodiment, second layer 110 is a conductive polymer, such as PEDOT. In another embodiment, second layer 110 is a nano gold coating that is applied via inkjet printing. In such case, printing allows exact dimensions so that no masking is needed in forming second layer 110. This allows for the use of automation in manufacturing, which leads to significant cost savings in production.

As with previous illustrations, first and second contact areas 102a and 102b are located on substrate 102 at the respective ends of layered structure 120, and will be accessible for attachment of sensors or other devices. In addition, just as all the embodiments above, further layers can be readily added to layered structure 120 such that 2, 3, 4 or more contact areas with respective conductive layers are readily available for coupling sensors, measuring devices, wires or other conductive contacts useful in invasive or implantable medical devices.

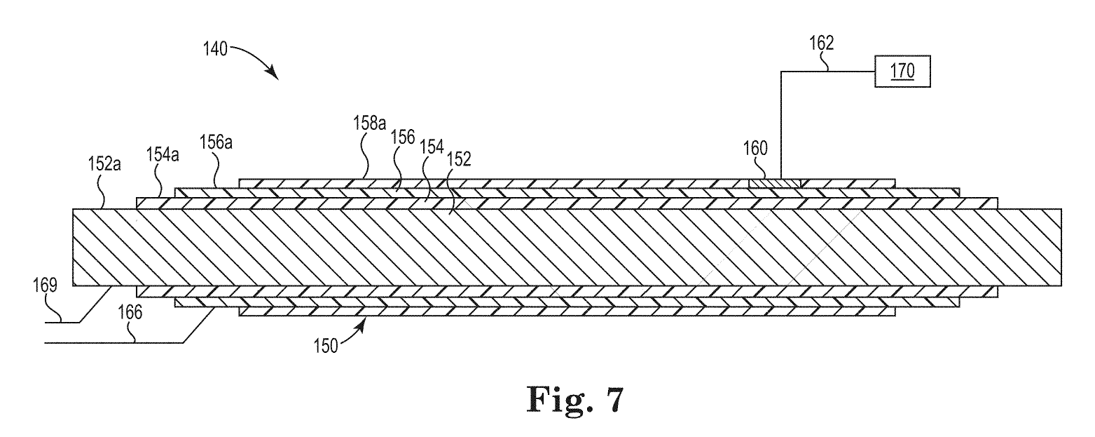

For example, FIG. 7 illustrates an implantable medical system 140 including a layered structure 150 according to one embodiment. Layered structure 150 includes substrate 152, which is conductive, first layer 154, which is insulating, second layer 156, which is conductive, and third layer 158, which is insulating. As with the above embodiments, the ends of each of substrate 152 and layers 154, 156, and 158 are slightly staggered relative to each other thereby defining contact area 152a for substrate 152 and contact area 156a for second layer 156.

In one embodiment of medical device 140, wire sensors 164 and 166 are respectively coupled to contact areas 152a and 156a. These wire sensors 164 and 166 may be wires, coils or other conductive elements that can receive signals, such as signals within a human body. Signals from wire sensors 164 and 166 are then respectively transmitted along substrate 152 and second layer 156. In one embodiment, it may be advantageous for a signal to be monitored or otherwise received by an electronic device 170. In some embodiments, the end portion opposite contact areas 152a and 156a may not be convenient for attachment to such a device. Accordingly contact area 160 is formed in second layer 158, such that coupling 162 can couple signals from second layer 156, through third layer 158 to electronic device 170.

This configuration is readily created using the exemplary layers described in the above embodiments. For example, second layer 158 could initially be formed using the conductive polymer CLEVIOS™. Next, the CLEVIOS™ Etch can be applied to the entire second layer 158 with the exception of the contact area 160. In this way, a conductive path is established between second layer 156 and electronic device 170. In another example, second layer 158 could be formed from a photolithographic process where the entire second layer 158 with the exception of the contact area 160 is photoresist, and contact area 160 is filled in with a conductive material.

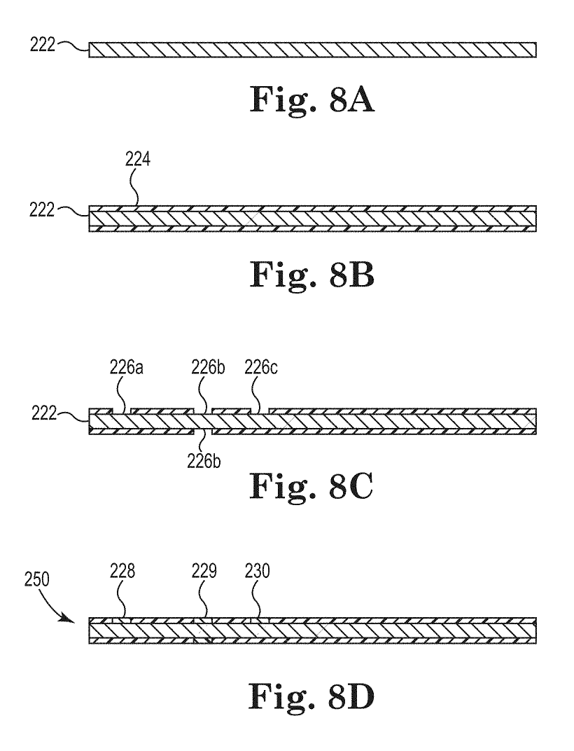

FIGS. 8a-8d illustrate sectional views of a method of forming a layered structure 250 (FIG. 8d) in accordance with one embodiment. A substrate 222 is illustrated in FIG. 8a. Substrate 222 is biocompatible and conductive, such as a medical wire. In FIG. 8b, first layer 224 is formed over substrate 222. In one embodiment, first layer 224 is a Poly(methyl methacrylate) (PMMA) coating that is a conductive insulator providing conductive isolation to the substrate 222. In one example, the PMMA coating of first layer 224 is dip coated to cover substrate 222.

Next, first, second and third access areas 226a, 226b and 226c are formed in first layer 224 such that substrate 222 is exposed through first layer 224 in those areas. In one embodiment, first access area 226a exposes just a small section of substrate 222 on a top surface and does not fully expose substrate 222 around its full circumference. In one embodiment, second access area 226b does fully expose substrate 222 around its full circumference. In other embodiments, additional or fewer access areas may be used to provide varying degrees of access to substrate 222. In one embodiment, first, second and third access areas 226a, 226b and 226c are formed by laser ablation of first layer 224 in those areas, although in other embodiments masking and other techniques as described above may be used.

In FIG. 8d, first, second and third contact areas 228, 229 and 230 are formed within first, second and third access areas 226a, 226b and 226c and directly over substrate 222. In one embodiment, first, second and third contact areas 228, 229 and 230 are formed with a conductive polymer, such as PEDOT. Such conductive polymers can be applied by dip coating or other various techniques described above. In such embodiments, masking as described above can be used to tailor the dimensions of contact areas 228/229/230 quite tightly. In other embodiments, first, second and third contact areas 228, 229 and 230 are formed with a nano gold coating that is applied via inkjet printing. In such case, printing allows exact dimensions so that no masking is needed in forming layered structure 250.

This allows for the use of automation in manufacturing, which leads to significant cost savings in production. In another embodiment, the configuration of layered structure 250 is achieved using CLEVIOS™ and CLEVIOS™ Etch with masking as discussed in the above embodiments.

The described embodiments of layered structure 250 can achieve very small contact areas 228/229/230 within first layer 224, which is itself very thin. For example, in one embodiment substrate 222 is a metal wire with a diameter of 0.5 mm and first layer 224 is an insulative polymer with a thickness of no more than 50 microns, and in one embodiment between 2 and 10 microns. In addition, contact areas 228/229/230 can achieve conductive contact with substrate 222 even with very small first, second and third access areas 226a, 226b and 226c. For example, first layer 224 can be laser ablated so that access areas 226a/226b/226c are just small diameter holes, in one embodiment having a diameter of less than 0.4 mm. In another embodiment, access areas 226a/226b/226c are more slit or rectangular shapes and have a width along substrate 222 that is 0.3 mm or less. Contact areas 228/229/230 can be formed exclusively within access areas 226a/226b/226c, or may even be formed to overlap first layer 224 slightly, but will still achieve conductive contact with substrate 222 as long as at least some portion of contact areas 228/229/230 are formed within access areas 226a/226b/226c.

As illustrated in FIG. 8d, contact areas 228/229/230 on layered structure 250, and can, for example, be located toward a distal end of layered structure 250. Signals can then be sent and/or received via substrate 222 at a proximal end of layered structure 250 to and from contact areas 228/229/230 at the distal end. In this way, when substrate 222 is a medical device configured for insertion into the human body, contact areas 228/229/230 can be used to measure signals within the body, deliver electrical stimulation to precise locations within the body or for similar uses of signals and electrical pulses, while access to these signals is provided at a location remote from where they are measured or delivered. Because both first layer 224 and contact areas 228/229/230 are very thin polymers, the overall size of layered structure 250 closes approximates substrate 222. Furthermore, the flexibility of these polymer materials ensures that layered structure 250 is no less flexible than is substrate 222.

In embodiments where contact areas 228/229/230 are dip coated within access areas 226a/226b/226c, the contact areas 228/229/230 can achieve very thin dimensions, yet at the same time provide a relatively consistent thickness across access areas 226a/226b/226c. This is not the case for sputter coating, which is common for applying thin metallic coatings. In such sputter coating processes, the device to be coated is placed in a chamber with essentially a cloud of atoms, such that everything in the chamber is coated with the conductive metallic material. In such environments, there is unevenness to the thickness of the applied layer due to the so-called “shadow effect.” With such a shadow effect, areas adjacent to vertical sections, such as the walls on either side of access areas 226a/226b/226c seen in FIG. 8c, are blocked by the vertical walls and are covered less than areas away from the side walls, such as in the center of access areas 226a/226b/226c. This unevenness of coverage caused by the shadow effect can cause problems with the integrity of the applied layer. This is not the case of the substantially even thickness layers of the dip-coated conductive layers.

For each of the layered structures illustrated in the above embodiments, the substrate is conductive. It is also possible in each case that the substrate is insulative. Generally, when the substrate is conductive, the first layer is then insulative, the second layer conductive and continuing alternating thereafter. When the substrate is insulative, the first layer is then conductive, the second layer insulative and continuing alternating thereafter. In each case, the substrate is generally a thicker layer that is useful in a medical device application, with each subsequent layer being an order of magnitude thinner than the substrate to essentially conform to the dimensions of the substrate.

It is also to be understood that the features of the various exemplary embodiments described above may be combined with each other, unless specifically noted otherwise. For example, masking techniques described in FIGS. 3a-3f could be used in forming some layers, while CLEVIOS™ Etch could be used in forming other layers, while laser ablation could be used in other layers, while photoresist techniques could be used in other layers, all in the same layered structure.

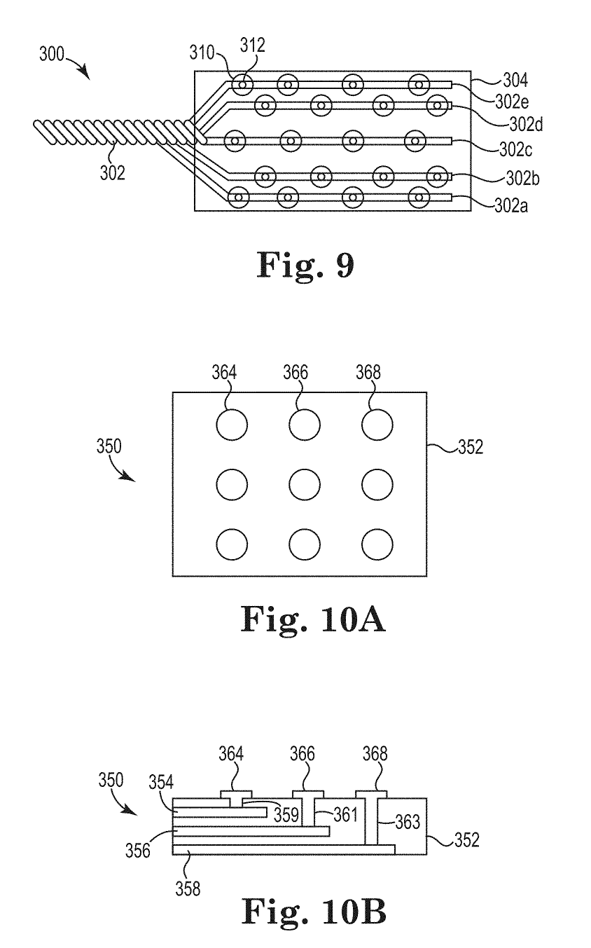

FIG. 9 illustrates an implantable medical system 300 including a multifilar layered structure 302 and electrode paddle 304 according to one embodiment. Multifilar layered structure 302 includes first-fifth layered structures 302a-302e, each of which are layered structures as described above with respect to the various embodiments and combinations thereof, and each of which are twisted together to form multifilar 302. In one embodiment, fifth layered structure 302e, for example, includes a wire substrate covered with insulating layer. Furthermore, access areas 312 (only labeled in one instance for simplifying the figure) have been laser ablated into the insulating layer and contact areas 310 (also only labeled in one instance for simplifying the figure) have been formed at least partially within access areas 312 (such as with the process described with respect to FIGS. 8A-8D above). As such, each of contact areas 310 are electrically coupled to the wire substrate. Each of contact areas 310 are then oriented on a face of electrode paddle 304. Accordingly, the various contact areas 310 of electrode paddle 304 can be energized independently by selectively energizing one or more of first-fifth layered structures 302a-302e.

In one embodiment, implantable medical system 300 is completely biocompatible such that it can be partially or completely implanted in a human body. Electrode paddle 304 can be located adjacent tissue within the body that will receive electrical energy via one or more contact areas 310. Electrical pulses can then be delivered to various locations on the tissue depending on which layered structures 302a-302e, and accordingly, which contact areas 310, are energized. Because contact areas 310 are distributed about electrode paddle 304 such that they are spaced apart from each other, certain areas of tissue or the like can be targeted by selectively energizing layered structures 302a-302e.

Similarly, FIGS. 10a and 10b illustrate an implantable medical system 350 including a multilayered structure 352 according to one embodiment. In one embodiment, multilayered structure 352 has a face with contact areas 364/366/368 (and other contact areas are illustrated without labels to simplify the figure). Each contact area is electrically coupled to one conductive layer 354/356/358. For example, first conductive layer 354 is coupled to first contact area 364, second conductive layer 356 is coupled to second contact area 366, and third conductive layer 358 is coupled to third contact area 368. Additional contact areas and corresponding conductive layers can be added as desired.

In each case, respective conductive layers 354/356/358 are electrically coupled to contact areas 364/366/368 with contact vias 359/361/363. The remaining portions of multilayered structure 352 are insulative so that each electrical signal on first conductive layer 354 is coupled only to first contact area 364, each electrical signal on second conductive layer 356 is coupled only to second contact area 366, and electrical signal on third conductive layer 358 is coupled only to third contact area 368. Each of conductive layers 354/356/358 and contact areas 364/366/368 can be constructed as described above with respect to FIGS. 8A-8D or any of the other various embodiments, and combinations thereof.

In one embodiment, implantable medical system 350 is completely biocompatible such that it can be partially or completely implanted in a human body. Just as with electrode paddle 304 above, the face of multilayered structure 352 can be located adjacent tissue within the body that will receive electrical energy via contact areas 364/366/368. Electrical pulses can then be delivered to various locations on the tissue depending on which conductive layers 354/356/358, and accordingly, which contact areas 364/366/368, are energized.

Many such useful medical systems can be constructed using the layer structures according to various embodiments herein. Although the above examples discuss a substrate that is generally cylindrical, such as a wire or a needle, other configurations are also possible. A substrate can have any geometrical shape that is known to, and deemed to be suitable for use in various medical applications. For example, a planar or arced surface such as, for example, a planar or arced plate, a planar or arced disc or a straight or curved tube may be used. In one embodiment, the substrate is coated according to the specified method on at least one surface of the substrate or on multiple surfaces, or on all surfaces, of the substrate. Different surfaces of the substrate can be provided with different or identical layered structures. In one embodiment, all surfaces of the substrate are simultaneously subjected to the method according to one embodiment in a single step. In this case, all surfaces treated according to the method have the same layered structure. The method according one embodiment is carried out in discontinuous manner as an immersion procedure. The method according to one embodiment can just as well be carried out continuously as a continuous system.

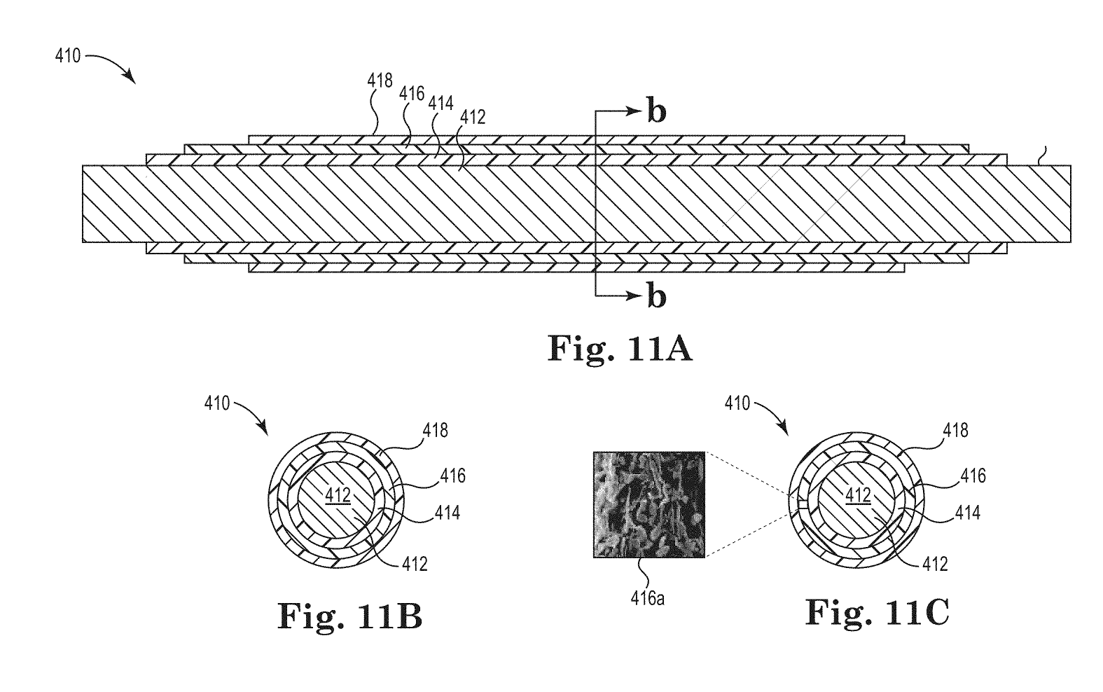

FIG. 11a illustrates a sectional view of layered structure 410 taken along its length, in accordance with one embodiment. FIGS. 11b and 11c illustrate sectional views of layered structure 410 from the line b-b in FIG. 11a. Layered structure 410 includes substrate 41, first layer 414, second layer 416 and third layer 418.

In one embodiment, first and third layers 414 and 418 isolate conductive substrate 412 and conductive second layer 416 so that independent signals can be carried via layered structure 410 from one end of layered structure 410 to the other end via substrate 412 and via second layer 416. Because layers 414, 416, and 418 are very thin and flexible, layered structure 410 significantly maintains the base shape, size, appearance and configuration of substrate 412.

In one embodiment, substrate 412 of layered structure 410 illustrated in FIGS. 11a-11c is a solid wire such as a medical guide wire or the like that can be implanted or temporality inserted into a human body. Alternating insulative and conductive layers 414, 416, and 418 are added such that layered structure 410 can also be used in medical implant situations.

FIG. 11b illustrates one embodiment where second layer 416 is a conductive compound made of conductive carbon material with an elastomer, or in one embodiment, with a polymer component. Such materials provide conductivity in second layer 416, while also maintaining both the thinness and the flexible of the layer. In some embodiments, the conductive carbon material can be provided as porous carbon particles. As such, when an elastomer, or in one embodiment a polymer, is added, the pores in these porous carbon particles are infiltrated with the elastomers or polymers to improve the mechanical stability of the carbon matrix inside the polymer.

In some alternative embodiments, such conductive carbons include carbon black, carbon nanotubes, graphene, and POROCARB®, a carbon component with high amount of macropores, sold by Heraeus. In some embodiments, polymers include a polyolefin (or polyethylene), a polyester, a poly silicone, epoxide resin, polyurethane, polystyrene, polyethylene terephthalate, polytetrafluoroethylene, and/and ethylene tetrafluoroethylene. In some embodiments, elastomers include Poly silicone (=silicone rubber), polybutadiene (=butadiene rubber), styrene-butadiene rubber, isoprene rubber, and/or fluroelastomers. Because these conductive carbons and polymers are biocompatible, layered structure 410 can also be used in medical implant situations.

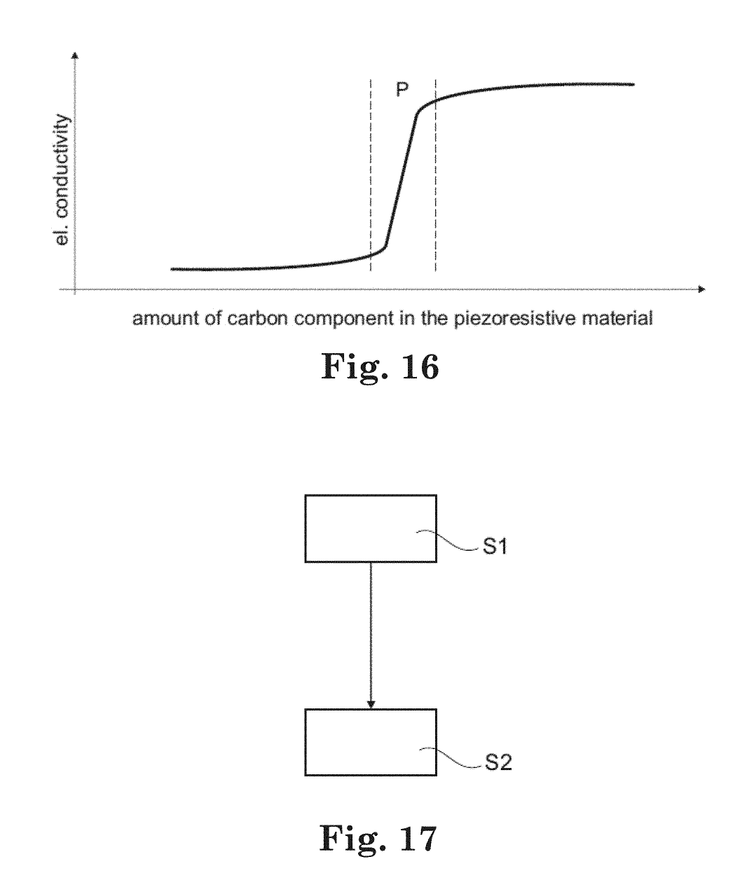

As will be discussed later below, FIG. 16 illustrates a schematic overview of the electrical conductivity of a type of conductive carbon material with an elastomer or polymer component, depending on a carbon concentration. The curve shows that above the area of the percolation threshold P, the electrical conductivity of the material is relatively high. Accordingly, as long as second layer 416 is a conductive compound made of conductive carbon material with an elastomer or polymer compound above the percolation threshold P, second layer 416 provides conductivity for layered structure 410, while maintaining thinness and flexibility.

FIG. 11c illustrates one embodiment where second layer 416 is a conductive compound made of conductive flakes with an elastomer or polymer component. As with prior embodiments, the conductive flakes with an elastomer or polymer component allows the second layer to be conductive, while also maintaining both the thinness and the flexible of the layer.

The exploded sectional view 416a in FIG. 11c illustrates the general orientation of the conductive flakes of second layer 416. As is apparent, the conductive flakes have relatively large length dimensions with relatively small height dimensions. Within layer 416, the conductive flakes tend to orient such that the longer length dimensions lay generally parallel to each other, or stack and overlap, generally on the tangent of the diameter of the first layer 414. In this way, even as layered structure 410 bends, the overlapping structure of the conductive flakes maintains the conductivity of layer 416. Although the conductive flakes can be of a variety of conductive materials, in one embodiment the conductive flakes are gold flakes such that they are biocompatible.

In other embodiments, conductive flakes of layer 416 are silver flakes. In one embodiment, the conductive silver flakes are formed as silver powder production via atomizing. In other embodiments, the silver flake production is via ball milling. In one embodiment, a mixing paste is formed with 60 weight percent silver flakes (0.5 μm thick 10 μm wide) with 30 weight percent polyurethane and 10 weight percent gamma-Butyrolactone. The paste is cured at 100° C. for 2 hours.

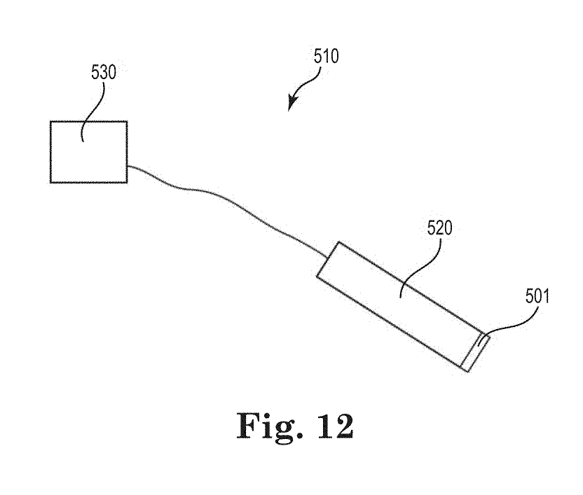

FIG. 12 illustrates schematically and exemplarily an embodiment of a detection unit 510 according to one embodiment. The detection unit 510 includes a detection element 520 and a processing element 530.

The processing element 530 may be an analog digital converter. The processing element 530 processes a decrease of electrical resistance detected by the piezoresistive material 501 into a value of compressive load applied to the piezoresistive material 501.

The detection element 520 may be a probe, a catheter tip, a blood pressure sensor, an artificial skin component or the like. The detection element 520 includes a piezoresistive material 501.

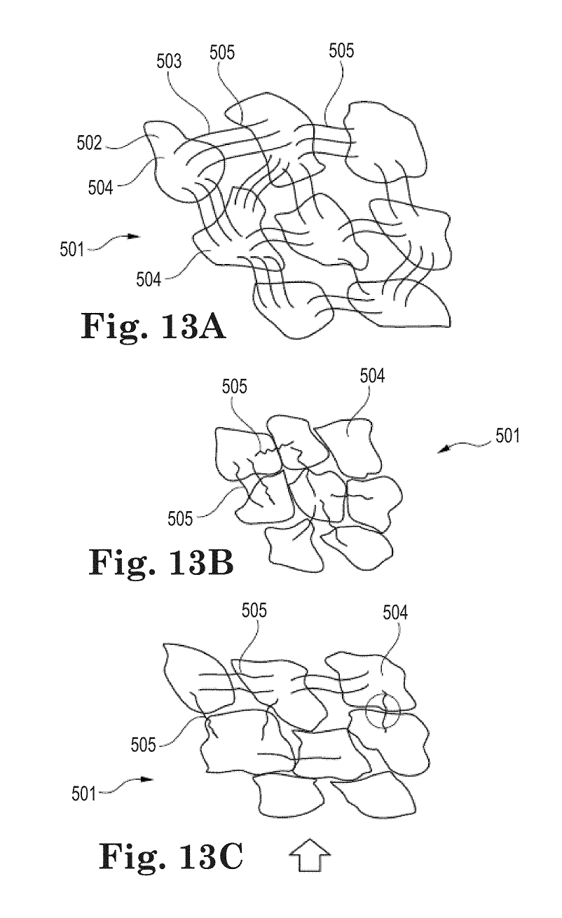

FIGS. 13a-13c illustrate schematically and exemplarily piezoresistive material 501 according to one embodiment. FIG. 13a illustrates the piezoresistive material 501 with no load or pressure. FIG. 13b shows illustrates piezoresistive material 501 with isostatic load or pressure. FIG. 13c illustrates the piezoresistive material 501 with uniaxial load or pressure.

As illustrated in FIG. 13a, the piezoresistive material 501 includes a compound of a carbon component 502 and an elastomer component 503. The carbon component 502 includes porous carbon particles 504, which includes macropores (not shown). The elastomer component 503 includes pre-stressed polymeric chains 505. Most of the macropores in the carbon particles 504 are infiltrated by polymeric chains 505. Further, most carbon particles 504 are linked by polymeric chains 505.

As illustrated in FIGS. 13b and 13c, the piezoresistivity of the piezoresistive material 501 is based on the fact that the polymeric chains 505 between the carbon particles 504 of the carbon component 502 rearrange and relax when the piezoresistive material 501 is subjected to a compressive load (isostatic in FIG. 13b, uniaxial in FIG. 13c). The rearrangement and relaxation enables a formation of electrical paths between the carbon particles 504 and consequently reduces the electrical resistance of the piezoresistive material 501.

The elastomer component 503 is here a silicone precursor.

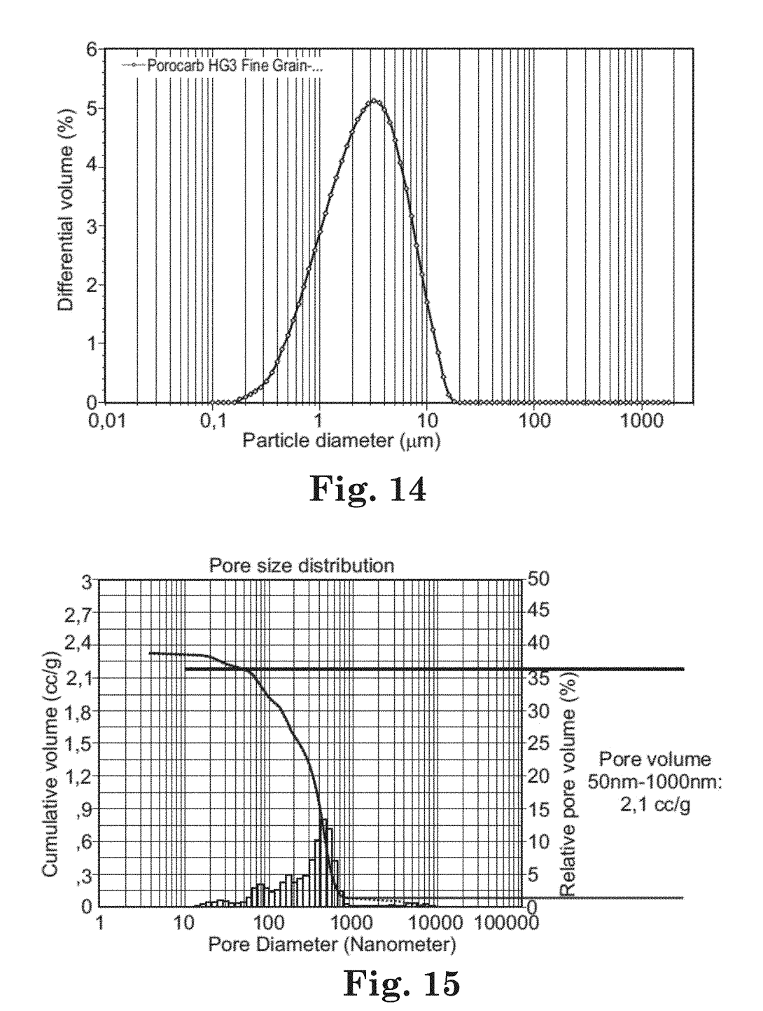

The carbon component 502 includes highly porous carbon particles 504 with open porosity. The pores of the carbon particles 504 include macropores with a size between 50 and 1000 nm. FIG. 14 illustrates a particle size distribution for an exemplary carbon component 502. The carbon particles 504 of the carbon component 502 are mainly between 1 and 20 μm. FIG. 15 illustrates a pore size distribution for an exemplary carbon particle 504. The total pore volume of the macropores is here 2.1 cm3/g and lies in general between 0.7 and 2.5 cm3/g. The carbon particles 504 further include mesopores with a size between 2 and 50 nm.

Here, only pores larger than a filling threshold between 60 and 250 nm are infiltrated by polymeric chains 505. Small macropores and mesopores are not filled. Micropores essentially do not exist due to a graphitization of the material.

The amount of the carbon component 502 in the material is near a percolation threshold P, which is here 18 wt.-%.

FIG. 16 illustrates a schematic overview of an electrical conductivity of the piezoresistive material 501 depending on a carbon concentration without external load. The curve illustrates within the area of the percolation threshold P a change of the electrical conductivity of the material. Below and above the area of the percolation threshold P, there is no sudden change of the electrical conductivity of the material.

When subjected to a load, the elastomer component 503 is compressed and thereby no longer blocks a contact between the actually electrically conductive carbon particles 504. Electrically conductive paths appear between the carbon particles 504. As the amount of the carbon component 502 in the piezoresistive material 501 is near the percolation threshold P, the appearance of the conductive paths leads to a sudden increase of the electrical conductivity of the piezoresistive material 501. The sudden increase of the electrical conductivity can be easily detected. As a result, near the percolation threshold P, the sensitivity for pressure is extremely high. Outside the area of the percolation threshold P, there is no sudden change of the electrical conductivity of the piezoresistive material 501.

FIG. 17 illustrates a schematic overview of steps of a method for producing a piezoresistive material 501 according to one embodiment. The method includes the following steps:
In a first step S1, mixing one or more elastomer components 503 and a carbon component 502 into a mixture, wherein the elastomer component 503 includes polymeric chains 505 and the carbon component 502 includes carbon particles 504 comprising macropores.
In a second step S2, curing the mixture so that at least some of the macropores in the carbon particles 504 are infiltrated by polymeric chains 505 to form a piezoresistive interconnection between the carbon particles 504.

Step S1 may also include a mixture with a curing agent. The curing agent may also be an elastomer component.

### Examples

As elastomer component, the two-component silicon Elastosil LR 3003/10 (Wacker Chemie AG) is used. Both subcomponents of Elastosil LR 3003/10 are liquid and highly viscous (η=74.000 mPa*s).

As carbon component, the macroporous carbon Porocarb HG3 Fine Grain (Heraeus) is used. Porocarb HG3 Fine Grain has a specific surface of 57 m2/g and a particle size d50 of 4 μm.

The carbon component is dispersed in both subcomponents of the elastomer component separately. This is done by means of a roller mill. Both subcomponents filled by the carbon component are then mixed with a 1:1 ratio to obtain the piezoresistive material.

The piezoresistive material is formed into a plate- and a rod-shape and cured in an oven for 4 hours at 200° C.

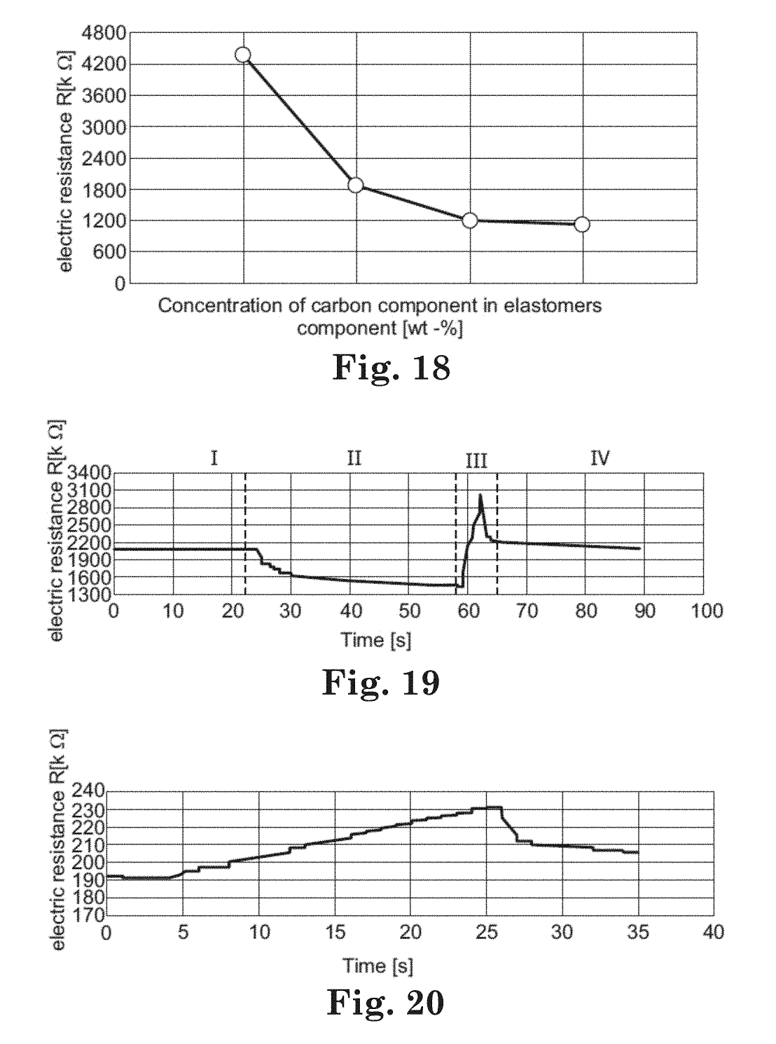

To detect a percolation threshold, several samples of the piezoresistive material with different concentrations of carbon particles are made and their electric conductivity is measured without any external force/pressure. The result is shown in FIG. 18. The electric conductivity is detected starting at a carbon particle concentration of 18 wt.-%. A maximum change of electric resistance (2503 kΩ) is detected for a carbon particle concentration between 18 wt.-% and 19 wt.-%. Starting at a carbon particle concentration of 21 wt.-%, no considerable change of the electric resistance is detected.

The samples of the piezoresistive material are subjected to unidirectional and isostatic pressure tests. Unidirectional pressure tests are made by means of a compression die. Isostatic pressure tests are made by means of a pressure chamber. The electric resistance is monitored by a multimeter (for example, Agilent 34401a).

FIG. 19 illustrates the detected electric resistance for an increasing load. Area I shows the electric resistance before the application of a load. In area II, the application of a load starts and the electric resistance decreases. The negative change of electric resistance for a load between 0 and 4 N amounts to 614 kΩ. Area III illustrates a peak when unloading the sample and a decrease of the electric resistance after unloading the sample. Area IV shows the return of the electric resistance to its initial value.

FIG. 20 illustrates a detected electric resistance for a sudden increase of blood pressure. The sudden increase of blood pressure leads to a considerable change of the detected electric resistance of the piezoresistive material, which thereby illustrates a great sensitivity for pressure changes.

As elastomer component, also the latex emulsion dispersion Lanxess S-62F can be used. Lanxess S-62F includes 68 wt.-% of styrene butadiene rubber and has a nominal density of 0.94 g/cm³. As carbon component, the carbon modification Porocarb HG3 Fine Grain (Heraeus) can be used again.

In a further example, 210 gr Porocarb HG-3FG are added to 1162 gr Lanxess S-62F to obtain a carbon concentration of 21 wt.-%. The mixture is agitated for 15 min. During further agitation, 70 gr diluted sulfuric acid (pH 3) with 1.4 gr of a polymer quaternary amine (for example, Perchem 503) are added at 60° C. SBR latex particles coagulate and precipitate. The liquid phase is separated by centrifugation.

As a result, an SBR rubber compound material is obtained. It is further agitated by a Brabender mixer B50 up to a temperature of 100° C. and cooled to 50° C. 2.5 gr Dicumyl peroxide (Sigma-Aldrich) are added to initiate cross-linking. The mixture is again agitated at 60° C. in the Brabender mixer, removed from the mixer and formed to samples to be tested as described above.

Although specific embodiments have been illustrated and described herein, it will be appreciated by those of ordinary skill in the art that a variety of alternate and/or equivalent implementations may be substituted for the specific embodiments illustrated and described without departing from the scope of the invention. This application is intended to cover any adaptations or variations of the specific embodiments discussed herein. Therefore, it is intended that this invention be limited only by the claims and the equivalents thereof.

What is claimed is:

1. A layered structure comprising:
a substrate;
a first layer over the substrate; and
a second layer over the first layer;
wherein the substrate and the second layer are an electrically conductive material and the first layer is an insulating material, or the substrate and the second layer are insulating material and the first layer is electrically conductive material, wherein each of the first and second layers are one tenth of the thickness of the substrate such that the layered structure substantially maintains the size and configuration of the substrate; and
wherein at least one of the first and second layers comprises a conductive carbon with polymer component.

2. The layered structure of claim 1, wherein the substrate and the first and second layers are biocompatible such that the layered structure is configured for implantation into the human body.

3. The layered structure of claim 1, wherein the conductive compound comprises a conductive carbon material above the percolation threshold.

4. The layered structure of claim 1, wherein the at least one of the first and second layers comprises a piezoresistive material.

5. The layered structure of claim 1 characterized in that each of the first and second layers are one tenth of the thickness of the substrate.

6. The layered structure of claim 1, wherein the layered structure has a length defining first and second ends and wherein at least one of the first and second layers extends substantially the length of the layered structure and includes contact areas proximate to the first and second ends that are configured for attaching an electrically conducting contact.

7. The layered structure of claim 1, wherein the thickness of at least one of the first and second layers is in a range from 0.05 μm to 10 μm.

8. The layered structure of claim 1, wherein the thickness of the first and second layers are such that the layered structure significantly maintains the base shape, size, appearance and configuration of the substrate.

9. A layered structure comprising:
a substrate;
a first layer over the substrate; and
a second layer over the first layer;
wherein the substrate and the second layer are an electrically conductive material and the first layer is an insulating material, or the substrate and the second layer are insulating material and the first layer is electrically conductive material, wherein each of the first and second layers are one tenth of the thickness of the substrate such that the layered structure substantially maintains the size and configuration of the substrate; and
wherein at least one of the first and second layers comprises conductive flakes embedded in a polymer binder.

10. The layered structure of claim 9, wherein the substrate and the first and second layers are biocompatible such that the layered structure is configured for implantation into the human body.

11. The layered structure of claim 9, wherein the conductive flakes are oriented to overlap each other, such that conductive flakes maintain the conductivity of layer even as the layered structure bends.

12. The layered structure of claim 9, wherein the at least one of the first and second layers comprises a piezoresistive material.

13. The layered structure of claim 9 characterized in that each of the first and second layers are one tenth of the thickness of the substrate.

14. The layered structure of claim 9, wherein the layered structure has a length defining first and second ends and wherein at least one of the first and second layers extends substantially the length of the layered structure and includes contact areas proximate to the first and second ends that are configured for attaching an electrically conducting contact.

15. The layered structure of claim 9, wherein the thickness of at least one of the first and second layers is in a range from 0.05 μm to 10 μm.

16. The layered structure of claim 9 further comprising a further conductive layer comprising a conductive carbon with polymer component.
